SEQUENCE LISTING (30 sequences)

SEQ ID NO: 1 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 227 aa, Artificial Sequence
  variant human Fc-region of the IgG1 isotype with the mutations L234A, L235A
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 227 aa, Artificial Sequence
  variant human Fc-region of the IgG1 isotype with a hole mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 4 - protein, 227 aa, Artificial Sequence
  variant human Fc-region of the IgG1 isotype with a knob mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 227 aa, Artificial Sequence
  variant human Fc-region of the IgG1 isotype with a L234A, L235A and hole mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 227 aa, Artificial Sequence
  variant human Fc-region of the IgG1 isotype with a L234A, L235A and knob mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, Artificial Sequence
  variant human Fc-region of the IgG1 isotype with a P329G mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, Artificial Sequence
  variant human Fc-region of the IgG1 isotype with a L234A, L235A and P329G mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 227 aa, Artificial Sequence
  variant human Fc-region of the IgG1 isotype with a P239G and hole mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 227 aa, Artificial Sequence
  variant human Fc-region of the IgG1 isotype with a P329G and knob mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 227 aa, Artificial Sequence
  variant human Fc-region of the IgG1 isotype with a L234A, L235A, P329G and hole mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 227 aa, Artificial Sequence
  variant human Fc-region of the IgG1 isotype with a L234A, L235A, P329G and knob mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 229 aa, Artificial Sequence
  variant human Fc-region of the IgG4 isotype with a S228P and L235E mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 229 aa, Artificial Sequence
  variant human Fc-region of the IgG4 isotype with a S228P, L235E and P329G mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 229 aa, Artificial Sequence
  variant human Fc-region of the IgG4 isotype with a S228P and L235E mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 17 - protein, 229 aa, Artificial Sequence
  variant human Fc-region of the IgG4 isotype with a S228P, L235E and P329G mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 2 aa, Artificial Sequence
  peptide linker 1
GS

SEQ ID NO: 19 - protein, 3 aa, Artificial Sequence
  peptide llinker 2
GGS

SEQ ID NO: 20 - protein, 4 aa, Artificial Sequence
  peptide linker 3
GGGS

SEQ ID NO: 21 - protein, 5 aa, Artificial Sequence
  peptide linker 4
GGGGS

SEQ ID NO: 22 - protein, 445 aa, Artificial Sequence
  latency inducing anti-PAI-1 antibody heavy chain
QVQLVQSGAEVKKPGASVKVSCKASGYTFTNYGMNWVRQAPGQGLEWMGWINTYTGEPTYTDDFKGRFTMTLDTSISTAYMELSRLRSDDTAVYYCAKDVSGFVFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 220 aa, Artificial Sequence
  latency inducing anti-PAI-1 antibody light chain
DIVMTQSPDSLAVSLGERATINCKSSQSLLNIIKQKNCLAWYQQKPGQPPKLLIYWASTRESGVPDRFSGSGSGTDFTLTISSLQAEDVAVYYCQQYYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 15 aa, Homo sapiens
GTVASSSTAVIVSAR

SEQ ID NO: 25 - protein, 15 aa, Artificial Sequence
  latency inducing polypeptide
GTVASSSTAVIVSAS

SEQ ID NO: 26 - protein, 37 aa, Homo sapiens
ESCKGRCTEGFNVDKKCQCDELCSYYQSCCTDYTAEC

SEQ ID NO: 27 - protein, 30 aa, Artificial Sequence
  peptide linker 5
GGGGSGGGGSGGGGSGGGGSGGGGSGGGGS

SEQ ID NO: 28 - protein, 299 aa, Artificial Sequence
  PAI1-0004
GTVASSSTAVIVSARGGGGSGGGGSGGGGSGGGGSESCKGRCTEGFNVDKKCQCDELCSYYQSCCTDYTAECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 309 aa, Artificial Sequence
  PAI1-0005
GTVASSSTAVIVSARGGGGSGGGGSGGGGSGGGGSGGGGSGGGGSESCKGRCTEGFNVDKKCQCDELCSYYQSCCTDYTAECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 309 aa, Artificial Sequence
  PAI1-0035
GTVASSSTAVIVSASGGGGSGGGGSGGGGSGGGGSGGGGSGGGGSESCKGRCTEGFNVDKKCQCDELCSYYQSCCTDYTAECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2017/0008949 A1
(43) Pub. Date: Jan. 12, 2017

(54) Use of the binding domain of a subunit of a multi-subunit structure for targeted delivery of pharmaceutically active entities to the multi-subunit structure

(71) Applicant: Hoffmann-La Roche Inc., Little Falls, NJ (US)

(72) Inventors: Guy Georges, Habach (DE); Marcel Gubler, Arlesheim (CH); Sabine Imhof-Jung, Planegg (DE)

(73) Assignee: Hoffmann-La Roche Inc., Little Falls, NJ (US)

(21) Appl. No.: 15/178,247

(22) Filed: Jun. 9, 2016

Related U.S. Application Data
(63) Continuation of application No. PCT/EP2014/076952, filed on Dec. 9, 2014.

(30) Foreign Application Priority Data
Dec. 10, 2013 (EP) 13196356.3

(51) Int. Cl. C07K 14/78; A61K 38/57; A61K 38/39; C07K 14/81

(52) U.S. Cl. CPC C07K 14/78; C07K 14/8132; A61K 38/57; A61K 38/39; C07K 2319/30; C07K 2319/70

(57) ABSTRACT

Herein is reported the use of a conjugate of a subunit of a multi-subunit structure and one biologically active entity for targeted delivery of the biologically active entity to the multi-subunit structure.

### CROSS REFERENCE TO RELATED APPLICATIONS

This application is a continuation of International Application No. PCT/EP2014/076952, having an international filing date of Dec. 9, 2014, the entire contents of which are incorporated herein by reference, and which claims benefit under 35 U.S.C. §119 to European Patent Application 13196356.3, filed Dec. 10, 2013.

### SEQUENCE LISTING

This application hereby incorporates by reference the material of the electronic Sequence Listing filed concurrently herewith. The material in the electronic Sequence Listing is submitted as a text (.txt) file entitled “P31358-US Sequence Listing ST25” created on Jun. 2, 2016, which has a file size of 51 kilo bytes, and is herein incorporated by reference in its entirety.

Herein is reported a method for targeted delivery of a pharmaceutically active entity directly to its site of action on a multi-subunit structure by using the binding domain of a subunit of the multi-subunit structure as a targeting and payload delivering entity.

### BACKGROUND OF THE INVENTION

In WO 2002/24219 an isolated protein complex is reported which includes a growth factor, growth factor binding protein and vitronectin. Also reported are methods of modulating cell proliferation and/or migration by administering said protein complex for the purposes of wound healing, skin repair and tissue replacement therapy.

In WO 2009/033095 compositions of humanized anti-PAI-1 antibodies and antigen-binding fragments thereof which convert PAI-1 to its latent form are reported. Another aspect reported relates to antibodies which bind and neutralize PAI-1 by converting PAI-1 to its latent form or increasing proteolytic cleavage. Another aspect reported relates to the use of humanized antibodies which inhibit or neutralize PAI-1 for the detection, diagnosis or treatment of a disease or condition associated with PAI-1 or a combination thereof.

In WO 2009/131850 a method for treating glaucoma or elevated IOP in a patient comprising administering to the patient an effective amount of a composition comprising an agent that inhibits PAI-1 expression or PAI-1 activity is reported.

Many if not all approaches for targeted delivery have the drawback of species limitation, i.e. species cross-reactive approaches are hardly known e.g. for surrogate studies in experimental animals

Many if not all approaches for targeted delivery are specific for certain targets.

In WO 2009/089059 therapeutic inhibitors of PAI-1 function and methods of their use are reported. WO 2012/085076 reports uPAR-antagonists and uses thereof In WO 2012/035034 fusion polypeptides comprising a serpin-fingerpolypeptide and a second peptide, polypeptide or protein and the use of such polypeptides is reported.

### SUMMARY OF THE INVENTION

It has been found that a binding domain of a subunit of a multi-subunit structure, e.g. a multi-subunit protein, can be used for the targeted delivery of a therapeutically active entity, e.g. an inhibitory polypeptide, to the multi-subunit structure.

It has been found that the specific binding interaction of a binding domain derived from a subunit of a multi-subunit structure can be used for targeted delivery of a therapeutically active entity that has been conjugated to the binding domain.

The use and the method as reported herein are based on the exploitation of the specific binding interactions that exist between the individual subunits of a multi-subunit structure, especially their specific recognition characteristics. Although it would be possible to conjugate the therapeutically active entity to the full size subunit it is advantageous to reduce the size of the conjugate in order to allow recombinant production and application with acceptable doses. Thus, it is preferred to use only the binding domain of a subunit for proper recognition and targeting to the other subunits of the multi-subunit structure.

One aspect as reported herein is the use of a conjugate of a binding domain of a subunit of a multi-subunit structure and (exactly) one biologically active entity for targeted delivery of the biologically active entity to the multi-subunit structure.

In one embodiment the binding domain of the subunit can reversibly associate with and dissociate from the multi-subunit structure.

In one embodiment the binding domain is from the subunit that is the second largest subunit of the multi-subunit structure or the smallest subunit of the multi-subunit structure.

In one embodiment the multi-subunit structure is a two-subunit structure or a three-subunit structure or a four-subunit structure.

In one embodiment the multi-subunit structure is a multi-subunit protein, wherein at least the subunit or all individual subunits are non-covalently associated with each other.

In one embodiment the biologically active entity is a pharmaceutically active entity. In one embodiment the biologically active entity is a therapeutically active polypeptide.

In one embodiment the conjugate is a recombinant conjugate.

In one embodiment the conjugate further comprises a half-life prolonging entity. In one embodiment the half-life prolonging entity is selected from poly(ethylene glycol), human serum albumin or fragments thereof, and an antibody Fc-region.

In one embodiment the binding domain and the therapeutically active polypeptide and the half-life prolonging entity are, independently of each other, either conjugated directly or via a peptide linker to each other.

It has been found that in the conjugate as reported herein the potency of the single biologically active entity is sufficient to induce latency of PAI-1.

In one embodiment the conjugate comprises in N-terminal to C-terminal direction the biologically active entity and a binding domain of a subunit of a multi-subunit structure.

In one embodiment the conjugate further comprises an antibody Fc-region. In one embodiment the antibody Fc-region is at the C-terminus of the conjugate.

It has been found that the potency of the biologically active entity in the conjugate is improved when the human IgG heavy chain Fc-region is of IgG1 subclass and starts with aspartate at position 221 (corresponding to position 1 of SEQ ID NO: 01 to SEQ ID NO: 12) e.g. compared to human IgG heavy chain Fc-region starting with proline at position 217 (numbered according to Kabat EU index of human IgG1). In one embodiment a human IgG heavy chain Fc-region extends from Asp221 to the carboxyl-terminus of the heavy chain. In one preferred embodiment the heavy chain Fc-region has an amino acid sequence selected from the group consisting of SEQ ID NO: 01 to SEQ ID NO: 12.

In one embodiment the binding domain of a subunit of a multi-subunit structure is the SMB domain of vitronectin and the biologically active entity is the Reactive Center Loop (RCL) of PAI-1.

In one embodiment the conjugate comprises in N-terminal to C-terminal direction an SMB domain of vitronectin and one Reactive Center Loop (RCL) of PAI-1 and an antibody Fc-region.

One aspect as reported herein is a recombinantly produced conjugate of a binding domain of a subunit of a non-covalently associated multi-subunit protein and a biologically active polypeptide, characterized in that
the multi-subunit protein is a two-subunit protein and the subunit is the smaller subunit of the multi-subunit protein, or
the multi-subunit protein is a three-subunit protein and the subunit is the smallest or the second largest subunit of the multi-subunit protein, or
the multi-subunit protein is a four subunit protein and the subunit is the smallest or the second smallest or the second largest subunit of the multi-subunit protein.

One aspect as reported herein is a method for targeted delivery of a biologically active polypeptide to its site of action, characterized in that the site of action of the biologically active polypeptide is on a multi-subunit protein and (exactly) one biologically active polypeptide is conjugated to a binding domain of a subunit of a multi-subunit protein.

In one embodiment the binding domain of the subunit can reversibly associate with and dissociate from the multi-subunit protein.

In one embodiment the subunit is the second largest subunit of the multi-subunit protein or the smallest subunit of the multi-subunit protein.

In one embodiment the multi-subunit protein is a two-subunit protein or a three-subunit protein or a four-subunit protein.

In one embodiment at least the subunit or all individual subunits of the multi-subunit protein are non-covalently associated with each other.

In one embodiment the biologically active polypeptide is a therapeutically active polypeptide.

In one embodiment the conjugate is a recombinant conjugate.

In one embodiment the conjugate further comprises a half-life prolonging entity. In one embodiment the half-life prolonging entity is selected from poly(ethylene glycol), human serum albumin or fragments thereof, and an antibody Fc-region.

In one embodiment the binding domain and the therapeutically active polypeptide and the half-life prolonging entity are independently of each other either conjugated directly or via a peptide linker to each other.

### DESCRIPTION OF THE FIGURES

FIG. 1 General structure of a conjugate comprising the reactive center loop (RCL) of PAI-1, the SMB domain of vitronectin and a human Fc-region; 1: reactive center loop of PAI-1, 2: peptide linker, 3: SMB domain, 4: Fc-region.

FIG. 2 Mode of action of the conjugate as reported herein exemplified with a conjugate comprising the reactive center loop (RCL) of PAI-1, the SMB domain of vitronectin and a human Fc-region and the di-subunit structure of PAI-1 and vitronectin.

FIG. 3A Dose-response curve for the effect of construct PAI1-0001 on non-glycosylated human PAI-1.

FIG. 3B Dose-response curve for the effect of construct PAI1-0004 on non-glycosylated human PAI-1.

FIG. 3C Dose-response curve for the effect of construc PAI1-0036 on non-glycosylated human PAI-1.

FIG. 3D Dose-response curve for the effect of constructt PAI1-0046 on non-glycosylated human PAI-1.

FIG. 3E Dose-response curve for the effect of construct PAI1-0005 on non-glycosylated human PAI-1.

FIG. 4A Dose-response curves for the effect of constructt PAI1-0001 on glycosylated human PAI-1.

FIG. 4B Dose-response curves for the effect of constructt PAI1-0004 on glycosylated human PAI-1.

FIG. 4C Dose-response curves for the effect of constructt PAI1-0036 on glycosylated human PAI-1.

FIG. 4D Dose-response curves for the effect of constructt PAI1-0046 on glycosylated human PAI-1.

FIG. 4E Dose-response curves for the effect of constructt PAI1-0005 on glycosylated human PAI-1.

### DETAILED DESCRIPTION OF THE INVENTION

The use and the method as reported herein are based on the exploitation of the specific binding interactions that exist between the individual subunits of a multi-subunit structure, especially their specific recognition characteristics. Although it would be possible to conjugate the therapeutically active entity to the full size subunit it is advantageous to reduce the size of the conjugate in order to allow recombinant production and application with acceptable doses. Thus, it is preferred to use only the binding domain of a subunit for proper recognition and targeting to the other subunits of the multi-subunit structure.

The articles “a” and “an” are used herein to refer to one or to more than one (i.e., to at least one) of the grammatical object of the article. By way of example, “an antibody” means one antibody or more than one antibody.

The term “at least one” denotes one, two, three, four, five, six, seven, eight, nine, ten or more. The term “at least two” denotes two, three, four, five, six, seven, eight, nine, ten or more.

The term “biologically active entity” denotes an organic molecule, e.g. a biological macromolecule such as a peptide, polypeptide, protein, glycoprotein, nucleoprotein, mucoprotein, lipoprotein, synthetic polypeptide, or synthetic protein, that causes a biological effect when administered in or to artificial biological systems, such as bioassays using cell lines and viruses, or in vivo to an animal, including but not limited to birds or mammals, including humans. This biological effect can be but is not limited to enzyme inhibition or activation, binding to a receptor or a ligand, either at the binding site or circumferential, signal triggering or signal modulation. Biologically active polypeptides are without limitation for example immunoglobulins, or hormones, or cytokines, or growth factors, or receptor ligands, or agonists or antagonists, or cytotoxic agents, or antiviral agents, or imaging agents, or enzyme inhibitors, enzyme activators or enzyme activity modulators such as allosteric substances. In one embodiment the biologically active entity is a biologically active polypeptide. In one embodiment the biologically active polypeptide is a therapeutically active polypeptide. In one embodiment the therapeutically active polypeptide is a linear polypeptide and has a length of from 10 to 250 amino acid residues. In one embodiment the therapeutically active polypeptide has a length of from 10 to 100 amino acid residues. In one embodiment the therapeutically active polypeptide has a length of from 10 to 50 amino acid residues. In one embodiment the biologically active entity is a complete antibody light or heavy chain, or a scFv, or a scFab or a single domain antibody, or a single chain antibody.

The “conjugation” of a biologically active entity to a binding domain can be done by chemical means and recombinantly. For a recombinant conjugation the encoding nucleic acids of the biologically active entity and the binding domain are joined, either directly or with an intervening sequence encoding a linker peptide, contiguous and in reading frame. For chemical conjugation the biologically active entity and the binding domain can be conjugated by different methods, such as chemical binding, or binding via a specific binding pair. In one embodiment the chemical conjugation is performed by chemically binding via N-terminal and/or ε-amino groups (lysine), ε-amino groups of different lysins, carboxy-, sulfhydryl-, hydroxyl-, and/or phenolic functional groups of the amino acid sequence of the parts of the complex, and/or sugar alcohol groups of the carbohydrate structure of the complex. In one embodiment the biologically active entity is conjugated to the binding domain via a specific binding pair.

The term “Fc-region” herein is used to define a C-terminal region of an immunoglobulin heavy chain that contains at least a portion of the constant region. The term includes native sequence Fc-regions and variant Fc-regions. In one preferred embodiment a human IgG heavy chain Fc-region extends from Asp221 to the carboxyl-terminus of the heavy chain. However, the C-terminal lysine (Lys447) or the terminal glycine (Gly476) and lysine (Lys477) of the Fc-region may or may not be present. Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, Md. (1991), NIH Publication 91-3242. An “Fc-region” is a term well known and can be defined on basis of the papain cleavage of an antibody heavy chain. The conjugates as reported herein may comprise in one embodiment a human Fc-region or an Fc-region derived from human origin. In a further embodiment the Fc-region is either an Fc-region of a human antibody of the subclass IgG4 or an Fc-region of a human antibody of the subclass IgG1, IgG2, or IgG3, which is modified in such a way that no Fcγ receptor (e.g. FcγRIIIa) binding and/or no C1q binding can be detected. In one embodiment the Fc-region is a human Fc-region and especially either from human IgG4 subclass or a mutated Fc-region from human IgG1 subclass. In one embodiment the Fc-region is from human IgG1 subclass with mutations L234A and L235A. While IgG4 shows reduced Fcγ receptor (FcγRIIIa) binding, antibodies of other IgG subclasses show strong binding. However Pro238, Asp265, Asp270, Asn297 (loss of Fc carbohydrate), Pro329, Leu234, Leu235, Gly236, Gly237, Ile253, Ser254, Lys288, Thr307, Gln311, Asn434, or/and His435 are residues which, if altered, provide also reduced Fcγ receptor binding (Shields, R. L., et al., J. Biol. Chem. 276 (2001) 6591-6604; Lund, J., et al., FASEB J. 9 (1995) 115-119; Morgan, A., et al., Immunology 86 (1995) 319-324; EP 0 307 434). In one embodiment a conjugate as reported herein is in regard to Fcγ receptor binding of IgG4 subclass or of IgG1 or IgG2 subclass, with a mutation in L234, L235, and/or D265, and/or contains the PVA236 mutation. In one embodiment the mutations are S228P, L234A, L235A, L235E, and/or PVA236 (PVA236 denotes that the amino acid sequence ELLG (given in one letter amino acid code) from amino acid position 233 to 236 of IgG1 or EFLG of IgG4 is replaced by PVA). In one embodiment the mutations are S228P of IgG4, and L234A and L235A of IgG1. The Fc-region of an antibody is directly involved in ADCC (antibody-dependent cell-mediated cytotoxicity) and CDC (complement-dependent cytotoxicity). A complex which does not bind Fcγ receptor and/or complement factor C1q does not elicit antibody-dependent cellular cytotoxicity (ADCC) and/or complement dependent cytotoxicity (CDC).

A polypeptide chain of a wild-type human Fc-region of the IgG1 isotype has the following amino acid sequence:

(SEQ ID NO: 01)

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

A polypeptide chain of a variant human Fc-region of the IgG1 isotype with the mutations L234A, L235A has the following amino acid sequence:

(SEQ ID NO: 02)

DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

A polypeptide chain of a variant human Fc-region of the IgG1 isotype with a T366S, L368A and Y407V mutation has the following amino acid sequence:

(SEQ ID NO: 03)

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVCTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPGK.

A polypeptide chain of a variant human Fc-region of the IgG1 isotype with a T366W mutation has the following amino acid sequence:

(SEQ ID NO: 04)

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGK.

A polypeptide chain of a variant human Fc-region of the IgG1 isotype with a L234A, L235A and

T366S, L368A, Y407V mutation has the following amino acid sequence:

(SEQ ID NO: 05)

DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVCTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPGK.

A polypeptide chain of a variant human Fc-region of the IgG1 isotype with a L234A, L235A and T366W mutation has the following amino acid sequence:

(SEQ ID NO: 06)

DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGK.

A polypeptide chain of a variant human Fc-region of the IgG1 isotype with a P329G mutation has the following amino acid sequence:

(SEQ ID NO: 07)

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALGAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

A polypeptide chain of a variant human Fc-region of the IgG1 isotype with a L234A, L235A and P329G mutation has the following amino acid sequence:

(SEQ ID NO: 08)

DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALGAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

A polypeptide chain of a variant human Fc-region of the IgG1 isotype with a P239G and T366S, L368A, Y407V mutation has the following amino acid sequence:

(SEQ ID NO: 09)

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALGAPIEKTISKAKGQPREPQVCTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPGK.

A polypeptide chain of a variant human Fc-region of the IgG1 isotype with a P329G and T366W mutation has the following amino acid sequence:

(SEQ ID NO: 10)

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALGAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGK.

A polypeptide chain of a variant human Fc-region of the IgG1 isotype with a L234A, L235A, P329G and T366S, L368A, Y407V mutation has the following amino acid sequence:

(SEQ ID NO: 11)

DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALGAPIEKTISKAKGQPREPQVCTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPGK.

A polypeptide chain of a variant human Fc-region of the IgG1 isotype with a L234A, L235A, P329G and T366W mutation has the following amino acid sequence:

(SEQ ID NO: 12)

DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALGAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGK.

A polypeptide chain of a wild-type human Fc-region of the IgG4 isotype has the following amino acid sequence:

(SEQ ID NO: 13)

ESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

A polypeptide chain of a variant human Fc-region of the IgG4 isotype with a S228P and L235E mutation has the following amino acid sequence:

(SEQ ID NO: 14)

ESKYGPPCPPCPAPEFEGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

A polypeptide chain of a variant human Fc-region of the IgG4 isotype with a S228P, L235E and P329G mutation has the following amino acid sequence:

(SEQ ID NO: 15)

ESKYGPPCPPCPAPEFEGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLGSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

A polypeptide chain of a variant human Fc-region of the IgG4 isotype with a S228P, L235E, P329G and T366S, L368A, Y407V mutation has the following amino acid sequence:

(SEQ ID NO: 16)

ESKYGPPCPPCPAPEFEGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLGSSIEKTISKAKGQPREPQVYTLPPSQEEMTKNQVSLSCA
VKGFYPSDIAVEWESNGQPENNYKTTPPVLDSDGSFFLVSRLTVDKSRWQ
EGNVFSCSVMHEALHNHYTQKSLSLSLGK.

A polypeptide chain of a variant human Fc-region of the IgG4 isotype with a S228P, L235E, P329G and T366W mutation has the following amino acid sequence:

(SEQ ID NO: 17)

ESKYGPPCPPCPAPEFEGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLGSSIEKTISKAKGQPREPQVYTLPPSQEEMTKNQVSLWCL
VKGFYPSDIAVEWESNGQPENNYKTTPPVLDSDGSFFLYSRLTVDKSRWQ
EGNVFSCSVMHEALHNHYTQKSLSLSLGK.

The term “peptide linker” denotes amino acid sequences of natural and/or synthetic origin. It consists of a linear amino acid chain wherein the 20 naturally occurring amino acids are the monomeric building blocks. The peptide linker has a length of from 1 to 50 amino acids, in one embodiment between 1 and 28 amino acids, in a further embodiment between 2 and 25 amino acids. The peptide linker may contain repetitive amino acid sequences or sequences of naturally occurring polypeptides. The linker has the function to ensure that entities conjugated to each other can perform their biological activity by allowing the entities to be presented properly. In one embodiment the peptide linker is rich in glycine, glutamine, and/or serine residues. These residues are arranged e.g. in small repetitive units of up to five amino acids, such as GS (SEQ ID NO: 18), GGS (SEQ ID NO: 19), GGGS (SEQ ID NO: 20), and GGGGS (SEQ ID NO: 21). The small repetitive unit may be repeated one to five times. At the amino- and/or carboxy-terminal ends of the multimeric unit up to six additional arbitrary, naturally occurring amino acids may be added. Other synthetic peptide linkers are composed of a single amino acid, which is repeated between 10 to 20 times and may comprise at the amino- and/or carboxy-terminal end up to six additional arbitrary, naturally occurring amino acids. All peptide linkers can be encoded by a nucleic acid molecule and therefore can be recombinantly expressed. As the linkers are themselves peptides, the polypeptides connected by the linker are connected to the linker via a peptide bond that is formed between two amino acids.

The term “poly (ethylene glycol)” denotes a non-proteinaceous residue containing poly (ethylene glycol) as essential part. Such a poly (ethylene glycol) residue can contain further chemical groups which are necessary for binding reactions, which results from the chemical synthesis of the molecule, or which is a spacer for optimal distance of parts of the molecule. These further chemical groups are not used for the calculation of the molecular weight of the poly (ethylene glycol) residue. In addition, such a poly (ethylene glycol) residue can consist of one or more poly (ethylene glycol) chains which are covalently linked together. Poly (ethylene glycol) residues with more than one PEG chain are called multi-armed or branched poly (ethylene glycol) residues. Branched poly (ethylene glycol) residues can be prepared, for example, by the addition of polyethylene oxide to various polyols, including glycerol, pentaerythriol, and sorbitol. Branched poly (ethylene glycol) residues are reported in, for example, EP 0 473 084, U.S. Pat. No. 5,932,462. In one embodiment the poly (ethylene glycol) residue has a molecular weight of 20 kDa to 35 kDa and is a linear poly (ethylene glycol) residue. In another embodiment the poly (ethylene glycol) residue is a branched poly (ethylene glycol) residue with a molecular weight of 35 kDa to 40 kDa.

A “polypeptide” is a polymer consisting of amino acids joined by peptide bonds, whether produced naturally or synthetically. Polypeptides of less than about 20 amino acid residues may be referred to as “peptides,” whereas molecules consisting of two or more polypeptides or comprising one polypeptide of more than 100 amino acid residues may be referred to as “proteins.” A polypeptide may also comprise non-amino acid components, such as carbohydrate groups, metal ions, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

In one embodiment the biologically active entity is a therapeutically active polypeptide. The term “therapeutically active polypeptide” denotes a polypeptide which is tested in clinical studies for approval as human therapeutics and which can be administered to an individual for the treatment of a disease.

As known to a person skilled in the art, the use of recombinant DNA technology enables the production of numerous derivatives of a nucleic acid and/or polypeptide. Such derivatives can, for example, be modified in one individual or several positions by substitution, alteration, exchange, deletion, or insertion. The modification or derivatization can, for example, be carried out by means of site directed mutagenesis. Such modifications can easily be carried out by a person skilled in the art (see e.g. Sambrook, J., et al., Molecular Cloning: A Laboratory Manual, Cold Spring Harbor Laboratory Press, N.Y., USA (1999)). The use of recombinant technology enables a person skilled in the art to transform various host cells with exogenous (heterologous) nucleic acid(s). Although the transcription and translation, i.e. expression, machinery of different cells use the same elements, cells belonging to different species may have among other things a different so-called codon usage. Thereby identical polypeptides (with respect to amino acid sequence) may be encoded by different nucleic acid(s). Also, due to the degeneracy of the genetic code, different nucleic acids may encode the same polypeptide (see e.g. Sambrook, J., et al., Molecular Cloning: A Laboratory Manual, Cold Spring Harbor Laboratory Press, New York, USA (1999); Hames, B. D., and Higgins, S. J., Nucleic acid hybridization—a practical approach, IRL Press, Oxford, England (1985)).

Expression of a gene is performed either as transient or as permanent expression. The polypeptide(s) of interest are in general secreted polypeptides and therefore contain an N-terminal extension (also known as the signal sequence) which is necessary for the transport/secretion of the polypeptide through the cell wall into the extracellular medium. In general, the signal sequence can be derived from any gene encoding a secreted polypeptide. If a heterologous signal sequence is used, it preferably is one that is recognized and processed (i.e. cleaved by a signal peptidase) by the host cell. For secretion in yeast for example the native signal sequence of a heterologous gene to be expressed may be substituted by a homologous yeast signal sequence derived from a secreted gene, such as the yeast invertase signal sequence, alpha-factor leader (including Saccharomyces, Kluyveromyces, Pichia, and Hansenula α-factor leaders, the second described in U.S. Pat. No. 5,010,182), acid phosphatase signal sequence, or the C. albicans glucoamylase signal sequence (EP 0 362 179). In mammalian cell expression the native signal sequence of the protein of interest is satisfactory, although other mammalian signal sequences may be suitable, such as signal sequences from secreted polypeptides of the same or related species, e.g. for immunoglobulins from human or murine origin, as well as viral secretory signal sequences, for example, the herpes simplex glycoprotein D signal sequence. The DNA fragment encoding for such a pre segment is ligated in frame, i.e. operably linked, to the DNA fragment encoding a polypeptide of interest.

Polypeptides can be produced recombinantly in eukaryotic and prokaryotic cells, such as CHO cells, HEK cells and E.coli. If the polypeptide is produced in prokaryotic cells it is generally obtained in the form of insoluble inclusion bodies. The inclusion bodies can easily be recovered from the prokaryotic cell and the cultivation medium. The polypeptide obtained in insoluble form in the inclusion bodies has to be solubilized before purification and/or re-folding procedures can be carried out.

Different methods are well established and in widespread use for protein purification, such as affinity chromatography with microbial proteins (e.g. protein A or protein G affinity chromatography), ion exchange chromatography (e.g. cation exchange (sulfopropyl or carboxymethyl resins), anion exchange (amino ethyl resins) and mixed-mode ion exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (see e.g. Vijayalakshmi, M. A., Appl. Biochem. Biotech. 75 (1998) 93-102).

It has been found that a binding domain of a subunit of a multi-subunit structure, e.g. a multi-subunit protein, can be used for the targeted delivery of a therapeutically active entity, e.g. an inhibitory polypeptide, to the multi-subunit structure.

It has been found that the specific binding interaction of a binding domain derived from a subunit of a multi-subunit structure can be used for targeted delivery of a therapeutically active entity that has been conjugated to the binding domain.

One aspect as reported herein is the use of a conjugate of a binding domain of a subunit of a multi-subunit structure and a biologically active entity for targeted delivery of the biologically active entity to the multi-subunit structure.

In order to replace the naturally occurring subunit with the conjugate as reported herein preferably those multi-subunit structures can be targeted in which the subunits can reversibly associate and dissociate. Thus, in one embodiment the binding domain of the subunit can reversibly associate with and dissociate from the multi-subunit structure.

In order to not interfere with the overall association of the multi-subunit structure it is advantageous to choose the subunit from which the binding domain is derived to be as small as possible. In one embodiment the binding domain is from the subunit that is the second largest subunit of the multi-subunit structure or the smallest subunit of the multi-subunit structure.

In order to establish therapeutically relevant levels of the conjugate as reported herein in the circulation of a patient it is advisable to have a half-life in the range of days or weeks. Thus, in one embodiment the conjugate further comprises a half-life prolonging entity. In one embodiment the half-life prolonging entity is selected from poly(ethylene glycol), human serum albumin or fragments thereof, and an antibody Fc-region.

One aspect as reported herein is a recombinantly produced conjugate of a binding domain of a subunit of a non-covalently associated multi-subunit protein and a biologically active polypeptide, characterized in that
the multi-subunit protein is a two-subunit protein and the subunit is the smaller subunit of the multi-subunit protein, or
the multi-subunit protein is a three-subunit protein and the subunit is the smallest or the second largest subunit of the multi-subunit protein, or
the multi-subunit protein is a four subunit protein and the subunit is the smallest or the second smallest or the second largest subunit of the multi-subunit protein.

One aspect as reported herein is a method for targeted delivery of a biologically active polypeptide to its site of action, characterized in that the site of action of the biologically active polypeptide is on a multi-subunit protein and the biologically active polypeptide is conjugated to a binding domain of a subunit of a multi-subunit protein.

The invention is exemplified in the following with a conjugate comprising the reactive center loop of PAI-1 as therapeutically active polypeptide, the SMB domain of vitronectin as binding domain, and an Fc-region for half-life increase. This example does not represent a limitation of the scope of the herein reported method; it is merely present as an example of the concept as presented herein.

PAI-1 is a secreted 50 kDa glycoprotein that irreversibly inhibits two types of serine proteases involved in the plasminogen activation cascade, i.e. tissue plasminogen activator (tPA) and urokinase plasminogen activator (uPA). In this function, PAI-1 controls hemostasis (blood coagulation and fibrinolysis) as well as tissue remodeling (turnover and degradation of extracellular matrix). Moreover, when bound to vitronectin (VN), PAI-1 also inhibits activated protein C (APC), which is another serine protease that functions as a potent anticoagulant by interfering with the thrombin activation cascade. In addition to its anticoagulant activity, APC exerts a broad range of cyto-protective actions including suppression of inflammation, prevention of cell apoptosis and stabilization of endothelial barrier function.

In normal physiology, PAI-1 is expressed at low levels in renal tissue. However, under pathological conditions, PAI-1 synthesis by both resident kidney cells and infiltrating inflammatory cells occurs in acute and chronic human kidney diseases. We hypothesized that pharmacological inhibition of elevated PAI-1 activity could provide benefits in two ways: i) de-repression of plasminogen activation to induce more dynamic turnover of extracellular matrix in chronic fibrotic renal disease and ii) prevention of PAI-1-mediated APC inactivation to promote anti-inflammatory and cyto-protective functions, particularly in acute kidney injury.

The general underlying concept for the treatment of PAI-1-mediated diseases is to reduce the amount of active inhibitory PAI-1 by promoting the formation of the latent state and/or to inhibit vitronectin (VN) binding to PAI-1.

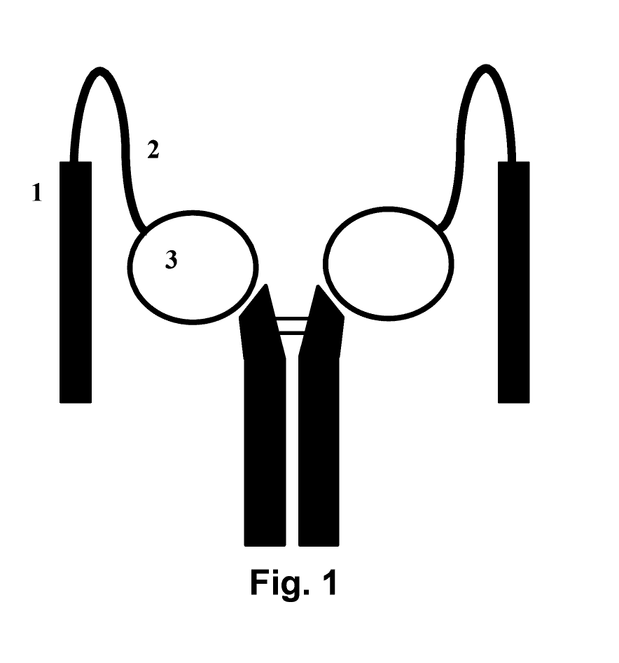

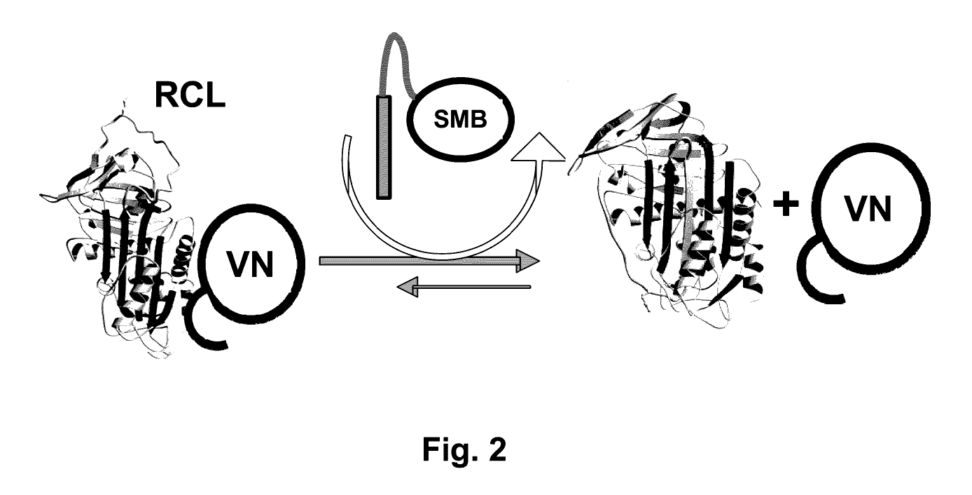

In order to promote the formation of the latent state, a conjugate comprising the reactive center loop (RCL) of PAI-1, the SMB domain of vitronectin and a human Fc-region has been generated. The general structure of this conjugate is shown in FIG. 1 and the mode of action is shown in FIG. 2.

For assessing the in vitro/in vivo efficacy of a conjugate according to the invention as reported herein a PAI-1 latency inducing antibody has been used (see e.g. US 2009/0081239). As no antibody-related effector functions are required/advisable, the antibody used was of the human IgG4 subclass with the mutation SPLE (S228P L235E). The reference antibody will be referred to in the following as PAI1-0001 in case of a murine IgG1 Fc-region and as PAI1-0046 in case of a human IgG4 SPLE Fc-region.

The amino acid sequence of the antibody heavy chain is:

(SEQ ID NO: 22)

QVQLVQSGAEVKKPGASVKVSCKASGYTFTNYGMNWVRQAPGQGLEWMGW
INTYTGEPTYTDDFKGRFTMTLDTSISTAYMELSRLRSDDTAVYYCAKDV
SGFVFDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFEGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK.

The amino acid sequence of the antibody light chain is:

(SEQ ID NO: 23)

DIVMTQSPDSLAVSLGERATINCKSSQSLLNIIKQKNCLAWYQQKPGQPP
KLLIYWASTRESGVPDRFSGSGSGTDFTLTISSLQAEDVAVYYCQQYYSY
PYTFGQGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC.

One aspect as reported herein is a latency inducing anti-human PAI-1 antibody that comprises the heavy chain CDRs of the heavy chain variable domain of SEQ ID NO: 22 and that comprises the light chain CDRs of the light chain variable domain of SEQ ID NO: 23.

In one embodiment the antibody comprises the heavy chain variable domain of SEQ ID NO: 22 and the light chain variable domain of SEQ ID NO: 23.

In one embodiment the antibody has an Fc-region of the human subclass IgG1 with the mutations L234A, L235A and optionally P329G.

In one embodiment the antibody has an Fc-region of the human subclass IgG4 with the mutations S228P, L235E and optionally P329G.

One aspect as reported herein is a recombinantly produced conjugate of the SMB domain of human vitronectin and a PAI-1 latency inducing polypeptide.

In one embodiment the latency inducing polypeptide has the amino acid sequence of GTVASSSTAVIVSAR (SEQ ID NO: 24).

In a preferred embodiment the latency inducing polypeptide has the amino acid sequence of GTVASSSTAVIVSAS (SEQ ID NO: 25).

In one embodiment the SMB domain has the amino acid sequence of ESCKGRCTEGFNVDKKCQCDELCSYYQSCCTDYTAEC (SEQ ID NO: 26).

In one embodiment the conjugate comprises a peptide linker between the latency inducing polypeptide and the SMB domain.

In one embodiment the peptide linker has a length of from 25 to 35 amino acid residues.

In one embodiment the peptide linker is (GGGGS)₆ (SEQ ID NO: 27).

In one embodiment the conjugate further comprises an antibody Fc-region.

In one embodiment the antibody Fc-region is of the human subclass IgG1 with the mutations L234A, L235A and optionally P329G.

In one embodiment the antibody Fc-region is of the human subclass IgG4 with the mutations S228P, L235E and optionally P329G.

In one embodiment the conjugate comprises in N- to C-terminal direction
a PAI-1 latency inducing polypeptide of SEQ ID NO: 24 or 25,
a peptide linker of SEQ ID NO: 27,
an SMB domain of SEQ ID NO: 26,
an antibody Fc-region of SEQ ID NO: 07 or 15.

In one embodiment the conjugate has the amino acid sequence of GTVASSSTAVIVSARGGGGSGGGGSGGGGSGGGGSESCKGRCTEGFNVDKKCQCDELC SYYQSCCTDYTAECDKTHTCPPCPAPELLGGPSVFLFPPKPKDTLMISRTPEVTCVVVDV SHEDPEVKFNWYVDGVEVHNAKTKPREEQYNSTYRVVSVLTVLHQDWLNGKEYKCK VSNKALPAPIEKTISKAKGQPREPQVYTLPPSRDELTKNQVSLTCLVKGFYPSDIAVEWE SNGQPENNYKTTPPVLDSDGSFFLYSKLTVDKSRWQQGNVFSCSVMHEALHNHYTQKS LSLSPGK (SEQ ID NO: 28). This conjugate is denoted in the following as PAI1-0004.

In one embodiment the conjugate has the amino acid sequence of GTVASSSTAVIVSARGGGGSGGGGSGGGGSGGGGSGGGGSGGGGSESCKGRCTEGFNV DKKCQCDELCSYYQSCCTDYTAECDKTHTCPPCPAPELLGGPSVFLFPPKPKDTLMISRT PEVTCVVVDVSHEDPEVKFNWYVDGVEVHNAKTKPREEQYNSTYRVVSVLTVLHQDW LNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEA LHNHYTQKSLSLSPGK (SEQ ID NO: 29). This conjugate is denoted in the following as PAI1-0005.

In one embodiment the conjugate has the amino acid sequence of GTVASSSTAVIVSASGGGGSGGGGSGGGGSGGGGSGGGGSGGGGSESCKGRCTEGFNV DKKCQCDELCSYYQSCCTDYTAECDKTHTCPPCPAPELLGGPSVFLFPPKPKDTLMISRT PEVTCVVVDVSHEDPEVKFNWYVDGVEVHNAKTKPREEQYNSTYRVVSVLTVLHQDW LNGKEYKCKVSNKALPAPIEKTISKAKGQPREPQVYTLPPSRDELTKNQVSLTCLVKGFY PSDIAVEWESNGQPENNYKTTPPVLDSDGSFFLYSKLTVDKSRWQQGNVFSCSVMHEA LHNHYTQKSLSLSPGK (SEQ ID NO: 30). This conjugate is denoted in the following as PAI1-0036.

The reference antibody and the conjugates as outlined above have been tested in a PAI-1 inhibition assay as outlined in Example 1. The determined IC₅₀-values against non-glycosylated and glycosylated human PAI-1 are shown in the following table.

IC₅₀ (μM) vs. human PAI-1

| Compound | non-glycosylated | glycosylated |
|---|---|---|
| PAI1-0001 | 0.007 | 0.116 |
| PAI1-0046 | 0.005 | 0.065 |
| PAI1-0004 | 0.003 | 0.002 |
| PAI1-0005 | 0.0005 | 0.002 |
| PAI1-0036 | 0.001 | 0.001 |

As can be seen, the conjugates according to the concept of the current invention are more potent latency-inducing (inhibiting) compounds compared to the reference antibody. Whereas the reference antibody shows a lower affinity (higher IC₅₀ value) to the glycosylated human PAI-1, the conjugates as reported herein shown a comparable affinity to both forms of human PAI-1, i.e. glycosylated and non-glycosylated.

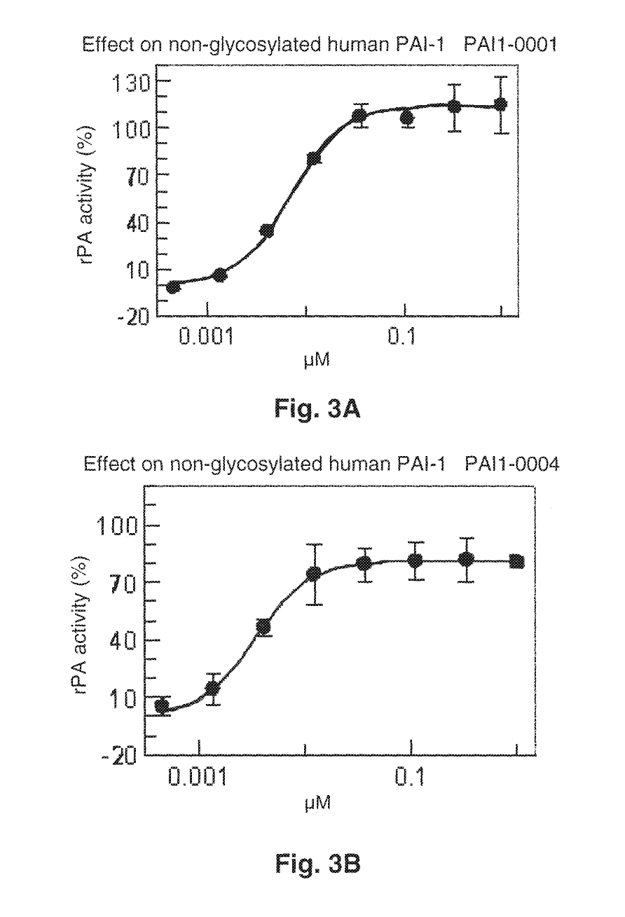

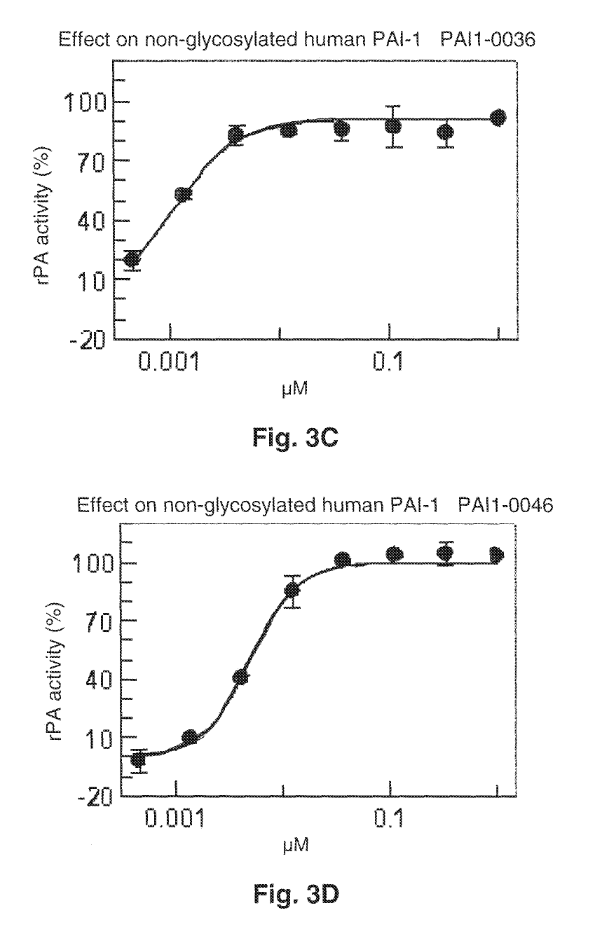

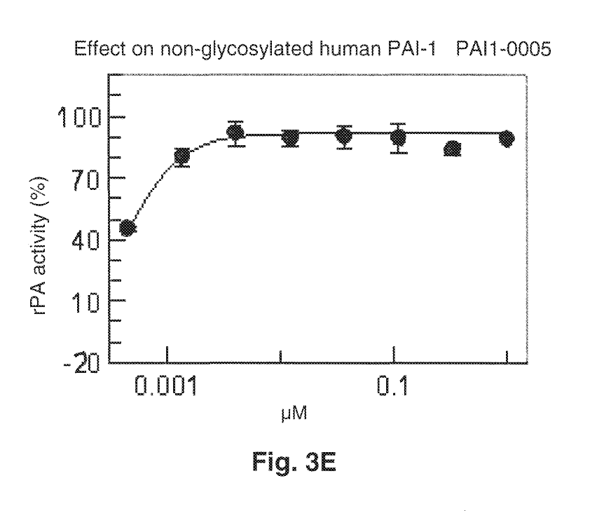

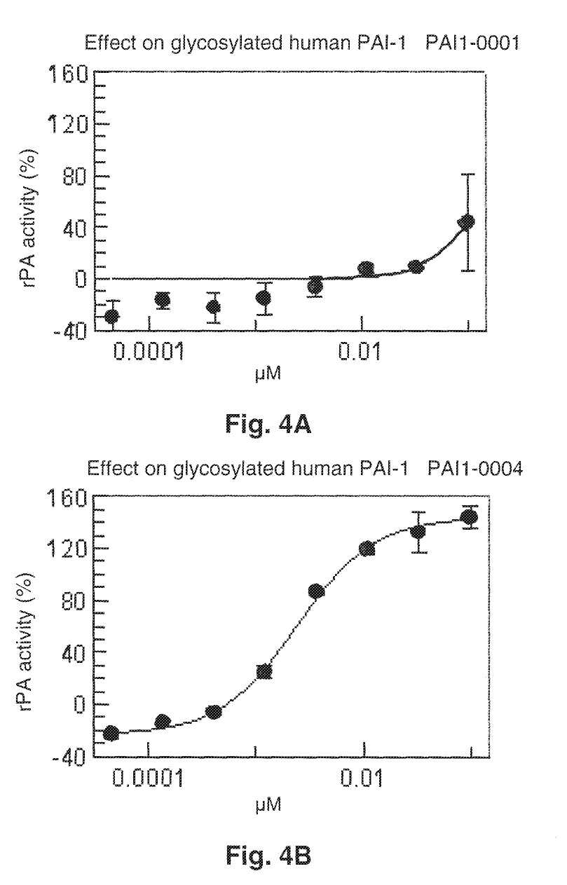

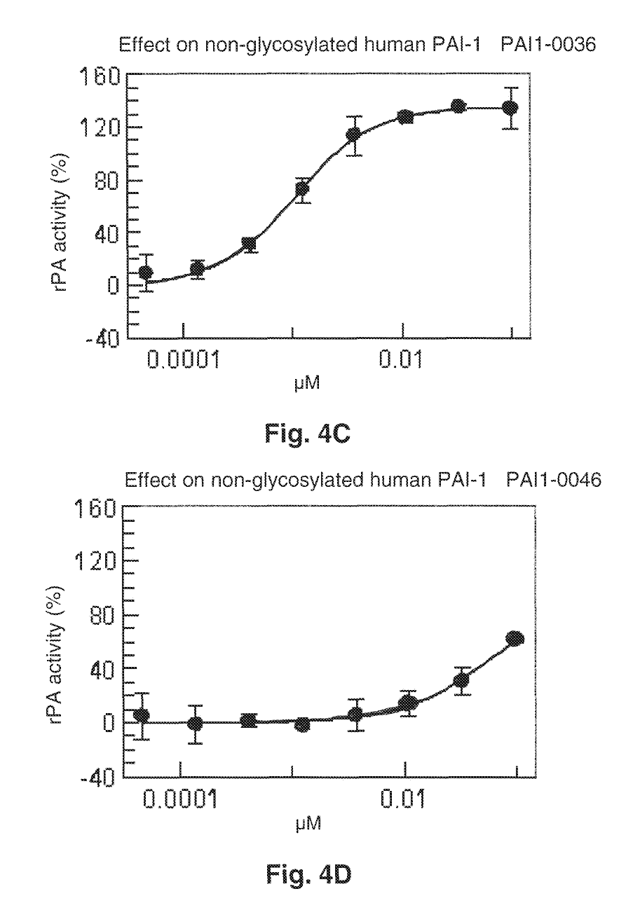

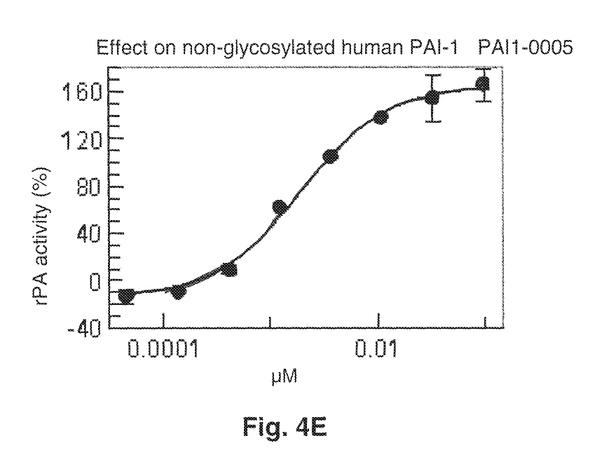

The corresponding dose-response curves are shown in FIGS. 3A-3E and 4A-4E.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

The following examples, sequences and figures are provided to aid the understanding of the present invention, the true scope of which is set forth in the appended claims. It is understood that modifications can be made in the procedures set forth without departing from the spirit of the invention.

### EXAMPLES

### Example 1

### Generation of Fusion Proteins

Recombinant DNA Techniques

Standard methods were used to manipulate DNA as described in Sambrook, J. et al., Molecular cloning: A laboratory manual; Cold Spring Harbor Laboratory Press, Cold Spring Harbor, N.Y., 1989. The molecular biological reagents were used according to the manufacturer's instructions.

Gene Synthesis

Gene synthesis fragments were ordered according to given specifications at Geneart (Regensburg, Germany). All gene segments encoding the RCL-SMB-Fc fusion proteins were synthesized with a 5′-end DNA sequence coding for a leader peptide (MGWSCIILFLVATATGVHS), which targets proteins for secretion in eukaryotic cells, and unique restriction sites at the 5′ and 3′ ends of the synthesized gene.

DNA Sequence Determination

DNA sequences were determined by double strand sequencing performed at Sequiserve GmbH (Vaterstetten, Germany).

DNA and Protein Sequence Analysis and Sequence Data Management

The GCG's (Genetics Computer Group, Madison, Wis.) software package version 10.2 and Infomax's Vector NT1 Advance suite version 11.0 was used for sequence creation, mapping, analysis, annotation and illustration.

Expression Vectors

For the expression of the described fusion molecules, expression plasmids for transient expression (e.g. in HEK293-F cells) based on a cDNA organization with a CMV-Intron A promoter were used.

Beside the antibody expression cassette the vectors contained:
an origin of replication which allows replication of this plasmid in E. coli, and
a B-lactamase gene which confers ampicillin resistance in E. coli.

The transcription unit of the antibody gene is composed of the following elements:
unique restriction site(s) at the 5′ end
the immediate early enhancer and promoter from the human cytomegalovirus,
followed by the Intron A sequence,
a 5′-untranslated region of a human antibody gene,
an immunoglobulin heavy chain signal sequence,
the gene for the fusion protein of RCL, SMB and human antibody IgG1 hinge and domains CH2 and CH3.
a 3′ untranslated region with a polyadenylation signal sequence, and
unique restriction site(s) at the 3′ end.

For transient and stable transfections larger quantities of the plasmids were prepared by plasmid preparation from transformed E. coli cultures (Nucleobond AX, Macherey-Nagel).

Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Transient Transfections in HEK293-F System

RCL-SMB-Fc fusion proteins were expressed by transient transfection of human embryonic kidney 293-F cells using the FreeStyle™ 293 Expression System according to the manufacturer's instruction (Invitrogen, USA). Briefly, suspension FreeStyle™ 293-F cells were cultivated in FreeStyle™ 293 Expression medium at 37° C./8% CO₂ and the cells were seeded in fresh medium at a density of 1-2×10⁶ viable cells/ml on the day of transfection. DNA-293fectin™ complexes were prepared in Opti-MEM® I medium (Invitrogen, USA) using 325 μl of 293fectin™ (Invitrogen, Germany) and 500 μg of plasmid DNA for a 250 ml final transfection volume. Fusion protein containing cell culture supernatants were harvested 7 days after transfection by centrifugation at 14000 g for 30 minutes and filtered through a sterile filter (0.22 μm). Supernatants were stored at −20° C. until purification.

Protein Determination

The protein concentration of purified fusion proteins was determined by determining the optical density (OD) at 280 nm, using the molar extinction coefficient calculated on the basis of the amino acid sequence according to Pace et. al., Protein Science, 1995, 4, 2411-1423.

Fusion Protein Concentration Determination in Supernatants

The concentration of fusion proteins in cell culture supernatants was measured by Protein A-HPLC chromatography. Briefly, cell culture supernatants containing fusion proteins that bind to Protein A were applied to a HiTrap Protein A column (GE Healthcare) in 50 mM K₂HPO₄, 300 mM NaCl, pH 7.3 and eluted from the matrix with 550 mM acetic acid, pH 2.5 on a Dionex HPLC-System. The eluted protein was quantified by UV absorbance and integration of peak areas. A purified standard IgG1 antibody served as a standard.

Purification of Fusion Proteins

Fusion proteins were purified from cell culture supernatants by affinity chromatography using Protein A-Sepharose™ (GE Healthcare, Sweden) and Superdex200 size exclusion chromatography. Briefly, sterile filtered cell culture supernatants were applied on a HiTrap ProteinA HP (5 ml) column equilibrated with PBS buffer (10 mM Na₂HPO₄, 1 mM KH₂PO₄, 137 mM NaCl and 2.7 mM KCl, pH 7.4). Unbound proteins were washed out with equilibration buffer. Fusion proteins were eluted with 0.1 M citrate buffer, pH 2.8, and the protein containing fractions were neutralized with 0.1 ml 1 M Tris, pH 8.5. Then, the eluted protein fractions were pooled, concentrated with an Amicon Ultra centrifugal filter device (MWCO: 30 K, Millipore) to a volume of 3 ml and loaded on a Superdex200 HiLoad 120 ml 16/60 gel filtration column (GE Healthcare, Sweden) equilibrated with 20mM Histidin, 140 mM NaCl, pH 6.0. Fractions containing purified fusion protein with less than 5% high molecular weight aggregates were pooled and stored as 1.0 mg/ml aliquots at −80° C.

SDS-PAGE

The NuPAGE® Pre-Cast gel system (Invitrogen) was used according to the manufacturer's instruction. In particular, 4-20% NuPAGE® Novex® TRIS-Glycine Pre-Cast gels and a Novex® TRIS-Glycine SDS running buffer were used. Reducing of samples was achieved by adding NuPAGE® sample reducing agent prior to running the gel.

Analytical Size Exclusion Chromatography

Size exclusion chromatography for the determination of the aggregation and oligomeric state of the fusion proteins was performed by HPLC chromatography. Briefly, Protein A purified fusion proteins were applied to a Tosoh TSKgel G3000SW column in 300 mM NaCl, 50 mM KH2PO4/K2HPO4, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

Mass Spectrometry

The total deglycosylated mass of fusion proteins was determined and confirmed via electrospray ionization mass spectrometry (ESI-MS). Briefly, 100 μg is purified fusion proteins were deglycosylated with 50 mU N-Glycosidase F (PNGaseF, ProZyme) in 100 mM KH₂PO₄/K₂HPO₄, pH 7 at 37° C. for 12-24 h at a protein concentration of up to 2 mg/ml and subsequently desalted via HPLC on a Sephadex G25 column (GE Healthcare). The mass of the reduced chain was determined by ESI-MS after deglycosylation and reduction. In brief, 50 μg antibody in 115 μl were incubated with 60 μl 1M TCEP and 50 μl 8 M Guanidine-hydrochloride subsequently desalted. The total mass and the mass of the reduced chain was determined via ESI-MS on a Q-Star Elite MS system equipped with a NanoMate source.

### Example 2

### PAI-1 Inhibition Assay

The method is based on the assay principle described by Lawrence et al. Eur. J. Biochem. 186 (1989) 523-533. A defined amount of active PAI-1 protein is mixed with a defined amount of a serine protease which is irreversibly blocked by active PAI-1. Residual serine protease activity is quantitatively determined by addition of a chromogenic peptide whose hydrolysis by the serine protease results in an increase in absorbance or fluorescence. Pre-incubation of active PAI-1 protein with defined concentrations of test compounds can result in latency induction (inhibition) of PAI-1. The degree of PAI-1 inhibition by test compounds is determined by measuring the proportional increase in serine protease activity (i.e. increase in absorbance or fluorescence). Use of serial dilutions of test compounds in this assay results in dose-response curves from which the potency of test compounds can be derived as IC50 values. The IC50 value represents the concentration of a test compound causing 50% inhibition of PAI-1 activity that is observed as 50% increase of serine protease activity. Typical PAI-1 inhibition assays are performed in black 96-well flat bottom micro-titer plates (Costar 3915) in a volume of 100 μl per well. All components including test compounds, active PAI-1, serine protease and chromogenic peptide are diluted in assay buffer (50 mM Tris-HCl pH 7.5 containing 150 mM NaCl, 0.01% Tween 80 and 0.1 mg/ml fatty acid-free BSA). In each well, 60 μl of assay buffer are mixed with 10 μl of 10-fold concentrated test compound and 10 μl of 10-fold concentrated active human PAI-1 protein (recombinant non-glycosylated human PAI-1, Roche batch #10_02, produced in E. coli as N-terminal 6×His-tagged fusion protein, 1 μg/ml; or recombinant glycosylated human PAI-1, Molecular Innovations product #GLYHPAI-A, produced in insect cells, 0.25 μg/ml). After incubation at 37° C. for 90 minutes, 10 μl of 10-fold concentrated serine protease are added (rPA=tPA deletion variant BM 06.022, Roche lot #PZ0606P064, batch #G366, 150 ng/ml). After incubation at 37° C. for 30 minutes, 10 μl of 10-fold concentrated chromogenic peptide are added (Spectrofluor tPA, American Diagnostica product #444F, 100 Fluorescence is measured in each well with a fluorescence plate reader (excitation at 358 nm, emission at 440 nm) immediately before and after an additional incubation of 2 hours at 37° C. The net increase in fluorescence intensity is calculated from the difference between fluorescence at t=2 hours minus fluorescence at t=0 hours. Control reactions without test compounds are included to define the dynamic range of the assay. Reactions with serine protease and with active PAI-1 protein represent the lower limit (0% rPA activity, 100% PAI-1 activity); reactions with serine protease but without PAI-1 protein represent the upper limit (100% rPA activity, 0% PAI-1 activity).

1-21. (canceled)

22. A protein conjugate comprising the somatomedin B (SMB) domain of human vitronectin and a single Plasminogen activator inhibitor-1 (PAI-1) latency inducing polypeptide.

23. The protein conjugate of claim 22, wherein the PAI-1 latency inducing polypeptide is a Reactive Center Loop (RCL) of PAI-1.

24. The protein conjugate of claim 23, where the RCL of PAI-1 is a 15-amino acid peptide fragment.

25. The protein conjugate of claim 22, wherein the PAI-1 latency inducing polypeptide comprises the amino acid sequence of SEQ ID NO: 24.

26. The protein conjugate of claim 22, wherein the PAI-1 latency inducing polypeptide comprises the amino acid sequence of SEQ ID NO: 25.

27. The protein conjugate of claim 22, wherein the SMB domain has the amino acid sequence of SEQ ID NO: 26.

28. The protein conjugate of claim 22, wherein the conjugate further comprises a peptide linker linking the PAI-1 latency inducing polypeptide and the SMB domain.

29. The protein conjugate of claim 28, wherein the peptide linker has a length of 25 to 35 amino acid residues.

30. The protein conjugate of claim 29, wherein the peptide linker has the amino acid sequence of SEQ ID NO: 27.

31. The protein conjugate of claim 22, wherein the conjugate further comprises an antibody Fc-region.

32. The protein conjugate of claim 31, wherein the antibody Fc-region is selected from:
a. the human immunoglobulin subclass IgG1 comprising mutations L234A and L235A; and,
b. the human immunoglobulin subclass IgG4 comprising mutations S228P, and L235E.

33. The protein conjugate of claim 22, wherein the conjugate comprises in N- to C-terminal direction:
a. a PAI-1 latency inducing polypeptide of SEQ ID NO: 24 or 25;
b. a peptide linker of SEQ ID NO: 27;
c. an SMB domain of SEQ ID NO: 26; and
d. an antibody Fc-region of SEQ ID NO: 07 or 15.

34. The protein conjugate of claim 33, wherein the conjugate has the amino acid sequence of SEQ ID NO: 28.

35. The protein conjugate of claim 33, wherein the conjugate has the amino acid sequence of SEQ ID NO: 29.

36. The protein conjugate of claim 33, wherein the conjugate has the amino acid sequence of SEQ ID NO: 30.

37. A protein conjugate comprising in N- to C-terminal direction: a PAI-1 latency inducing polypeptide and an SMB domain.

38. The protein conjugate of claim 37, wherein the PAI-1 latency inducing polypeptide is a RCL of PAI-1.

39. The protein conjugate of claim 38, where the RCL of PAI-1 is a 15-amino acid peptide fragment.

40. The protein conjugate of claim 37, further comprising an antibody Fc-region.

41. The protein conjugate of claim 40, wherein the conjugate comprises in N- to C-terminal direction: the PAI-1 latency inducing polypeptide, the SMB domain, and the antibody Fc region.

42. The protein conjugate of claim 37, wherein the PAI-1 latency inducing polypeptide comprises the amino acid sequence of SEQ ID NOs: 24 or 25.

43. The protein conjugate of claim 37, wherein the SMB domain has the amino acid sequence of SEQ ID NO: 26.

44. The protein conjugate of claim 37, wherein the conjugate further comprises a peptide linker of 25 to 35 amino acid residues linking the PAI-1 latency inducing polypeptide and the SMB domain.

45. The protein conjugate of claim 40, wherein the antibody Fc-region is selected from:
a. the human immunoglobulin subclass IgG1 comprising mutations L234A and L235A; and,
b. the human immunoglobulin subclass IgG4 comprising mutations S228P, and L235E.

46. The protein conjugate of claim 45, wherein the antibody Fc-region further comprises a P329G mutation.

47. A method of treating a PAI-1-mediated disease, comprising administering to a human subject in need of such treatment the protein conjugate of claim 22.

48. The method of claim 47, wherein the administration induces dynamic turnover of extracellular matrix in the subject.

49. The method of claim 47, wherein the administration promotes anti-inflammatory and cyto-protective functions by prevention of PAI-1-mediated activated protein C (APC) inactivation.

50. The method of claim 47, wherein the PAI-1-mediated disease is chronic fibrotic renal disease or acute kidney injury.

51. A method of treating a PAI-1-mediated disease, comprising administering to a human subject in need of such treatment the protein conjugate of claim 37.

52. The method of claim 51, wherein the administration induces dynamic turnover of extracellular matrix in the subject.

53. The method of claim 51, wherein the administration promotes anti-inflammatory and cyto-protective functions by prevention of PAI-1-mediated activated protein C (APC) inactivation.

54. The method of claim 51, wherein the PAI-1-mediated disease is chronic fibrotic renal disease or acute kidney injury.
